(19) United States
(12) Patent Application Publication
(10) Pub. No.: US 2026/0066039 A1
(43) Pub. Date: Mar. 5, 2026

(54) TRAINING AND USING GENERATIVE THERMODYNAMICS NEURAL NETWORKS TO DETERMINE BINDING AFFINITIES FROM ENERGY DISTRIBUTIONS

(71) Applicant: Recursion Pharmaceuticals, Inc., Salt Lake City, UT (US)

(72) Inventor: Oscar MENDEZ LUCIO, Madrid (ES)

(21) Appl. No.: 18/825,331

(22) Filed: Sep. 5, 2024

(51) Int. Cl. G16B 15/30; G16B 40/00

(52) U.S. Cl. CPC G16B 15/30; G16B 40/00

(57) ABSTRACT

The present disclosure relates to systems, non-transitory computer-readable media, and methods for training and utilizing generative thermodynamics neural networks to utilize an energy-to-base distribution transformation process to determine a binding conformation for a query compound and a target protein. For example, the disclosed systems can sample a conformation of the query compound from a known distribution and utilize the base-to-energy distribution transformation process to map the compound from the known distribution to a binding conformation. Moreover, the disclosed systems can determine an energy value associated with the binding conformation. In some bases, the disclosed systems can utilize an energy-to-base distribution transformation process to determine a binding metric for the binding conformation.

### BACKGROUND

Recent years have seen significant developments in hardware and software platforms for training and utilizing machine learning models in conjunction with computer-implemented pharmaceutical discovery systems. For example, conventional systems utilize large volumes of training data to teach machine learning models to generate intelligent predictions corresponding to complex biological interactions between genes, compounds, and/or proteins. Despite these recent advances, conventional systems suffer from a number of technical deficiencies, particularly with regard to accuracy, efficiency, and operational inflexibility in implementing machine learning technologies.

### SUMMARY

Embodiments of the present disclosure provide benefits and/or solve one or more of the foregoing or other problems in the art with systems, non-transitory computer readable media, and methods for training and utilizing generative thermodynamics machine learning models to predict binding affinities between the query compounds and target proteins. For example, the disclosed systems sample a conformation of a query compound from a known distribution of the query compound. The disclosed systems utilize a base-to-energy distribution transformation process to map the query compound from a known distribution (e.g., a Gaussian distribution) to an energy distribution (e.g., a Boltzmann distribution) and predict a binding conformation for the query compound and a target protein.

Moreover, in one or more embodiments, the disclosed systems can train a generative thermodynamics neural network to utilize a base-to-energy distribution transformation process to predict a binding conformation for a training compound and a training target protein. Specifically, the disclosed systems can determine an energy value corresponding to the binding conformation, and update parameters of the generative thermodynamics neural network according to the energy value. For example, the disclosed systems can compare an energy distribution (corresponding to binding conformation energy values) with a predicted probability distribution to teach the generative thermodynamics neural networks to map between energy distributions and base distributions. Indeed, by utilizing the base-to-energy distribution transformation process to predict the binding conformation and corresponding energy value, the disclosed systems can efficiently train generative thermodynamics neural networks to generate binding metrics without the need for excessive training data.

In addition, in one or more embodiments, the disclosed systems utilize an energy-to-base distribution transformation process comprising a generative thermodynamics neural network to generate a predicted series of conformations and corresponding conformation probabilities between the binding conformation and an unbound conformation of the query compound. The disclosed systems can utilize the corresponding conformation probabilities to generate a binding metric representative of a binding affinity between the query compound and the target protein. Moreover, in some instances, the disclosed systems can utilize the binding metric as a signal for analysis in conjunction with other models to generate a biological activity prediction.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates a thermodynamics binding system mapping a query compound from a known distribution to an energy distribution to determine a binding metric for the query compound and utilizing the binding metric in accordance with one or more embodiments.

FIG. 2 illustrates an example diagram of the thermodynamics binding system predicting a conformation change for a query compound.

FIG. 3 illustrates the thermodynamics binding system training a generative thermodynamics neural network to generate a binding conformation from a training compound and determine an energy value from the binding conformation in accordance with one or more embodiments.

FIG. 4 illustrate the thermodynamics binding system utilizing the generative thermodynamics neural network to generate a binding prediction for a query compound and determine a binding metric for the query compound from the binding conformation.

FIG. 5 illustrates the thermodynamics binding system utilizing a base-to-energy distribution transformation process to generate a binding conformation and utilizing an energy-to-base distribution transformation process to generate a binding metric from the binding conformation.

FIG. 6 illustrates an example graphical user interface of the thermodynamics binding system in accordance with one or more embodiments.

FIG. 7 illustrates an example environment of the thermodynamics binding system in accordance with one or more embodiments.

FIG. 8 illustrates an example series of acts for generating a binding metric in accordance with one or more embodiments.

FIG. 9 illustrates a block diagram of a computing device for implementing one or more embodiments.

### DETAILED DESCRIPTION

This disclosure describes one or more embodiments of a thermodynamics binding system 100 that trains and utilizes a generative thermodynamics neural network to generate a predicted binding metric for a query compound. For example, the thermodynamics binding system 100 utilizes a generative thermodynamics neural network in a base-to-energy distribution transformation process to generate a binding conformation corresponding to a binding interaction between the query compound and a target protein. Moreover, the thermodynamics binding system 100 can utilize the generative thermodynamics neural network in an energy-to-base distribution transformation process to generate a predicted series of conformations and corresponding conformation probabilities between the binding conformation and an unbound conformation of the query compound. Indeed, the thermodynamics binding system 100 can determine a binding metric representative of a binding interaction (e.g., a binding affinity) between the query compound and the target protein. The thermodynamics binding system 100 can utilize the binding metric in a variety of downstream applications. For instance, the thermodynamics binding system 100 can utilize the binding metric as an input into additional models, such as a compound program analysis.

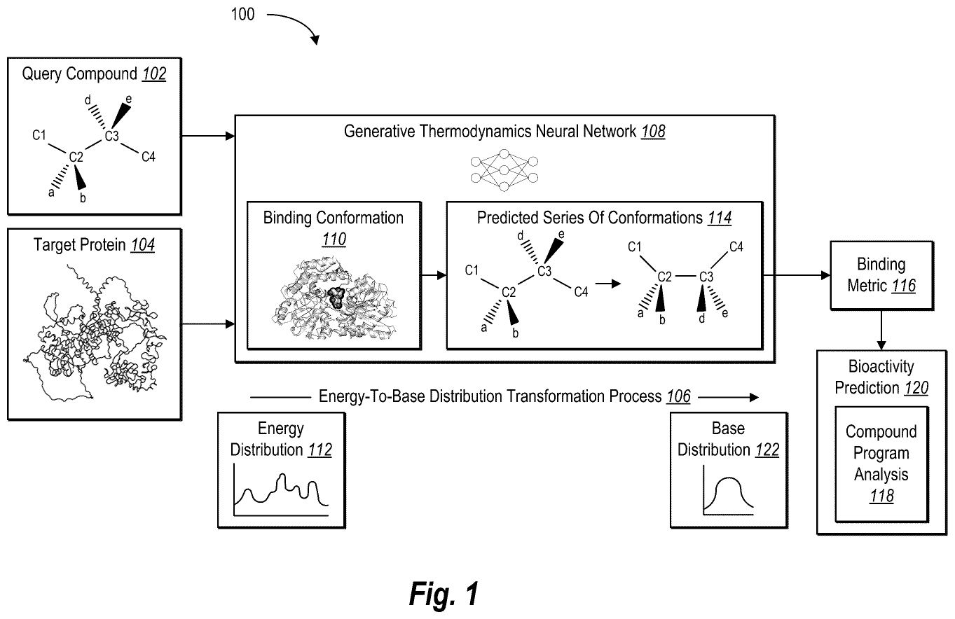

As just mentioned, the thermodynamics binding system 100 can utilize a generative thermodynamics neural network in a base-to-energy distribution transformation process to predict a binding conformation between a query compound and a target protein. From the binding conformation, the thermodynamics binding system 100 can utilize the generative thermodynamics neural network in an energy-to-base distribution transformation process to predict a binding metric representative of a binding affinity between the query compound and the target protein. For example, FIG. 1 illustrates the thermodynamics binding system 100 utilizing a generative thermodynamics neural network to determine a binding metric from a predicted binding conformation between a query compound and a target protein.

As shown in FIG. 1, the thermodynamics binding system 100 can receive, identify, and/or generate a query compound 102. Specifically, the thermodynamics binding system 100 can receive a binding query for the query compound 102 and a target protein 104. For instance, the thermodynamics binding system 100 can receive a query from a client device about a chemical interaction (e.g., a binding interaction) between a ligand (e.g., a query compound) and a target protein. For example, the thermodynamics binding system 100 can receive, identify, and/or generate a chemical formula or digital representation of the query compound 102 (and/or the target protein 104). To illustrate, the thermodynamics binding system 100 can receive a query from a client device identifying the query compound 102 and the target protein 104. The thermodynamics binding system 100 can then identify features of the query compound 102 and transform the query compound 102 (and/or the target protein 104) into a digital representation. For example, the thermodynamics binding system 100 can generate a structural representation of the query compound 102 and/or the target protein 104.

As illustrated in FIG. 1, the thermodynamics binding system 100 can utilize a generative thermodynamics neural network 108 to generate a bending metric from the query compound 102. Specifically, the thermodynamics binding system 100 can identify a binding conformation 110 and map the binding conformation 110 to a base distribution 122 utilizing the generative thermodynamics neural network 108 to determine the binding metric 116.

As shown in FIG. 1, the thermodynamics binding system 100 can identify or generate a binding conformation 110 of the query compound 102 and the target protein 104. As used herein, the term “binding conformation” refers to a conformation of the query compound 102 that corresponds to a chemical interaction (e.g., a binding interaction) between the initial conformation and the target protein 104. For example, the binding conformation 110 can represent a conformation of the query compound 102 resulting from a binding state between the query compound 102 and the target protein 104. Thus, the binding conformation 110 reflects a conformation from an energy distribution 112 (e.g., a Boltzmann distribution) indicating binding interactions between the query compound 102 and the target protein 104. Moreover, as used herein, the term “query compound” (or “compound”) refers to a molecule (e.g., a molecule provided by a computing device as part of a query regarding a target protein). A compound can include an existing, physical compound (e.g., a compound that has been synthesized) or an experimental, virtual, or synthetic compound (e.g., a compound that has not been physically synthesized). Moreover, compounds can include pharmaceutical compounds (e.g., small molecule usually less than 1000 Daltons that diffuse across cell membranes, such as alkaloids, antibiotics, steroids, vitamins, NSAIDs, among others). Additionally or alternatively, compounds can include other molecules (e.g., large molecules such as proteins, antibodies, nucleic acids, polysaccharides, glycoproteins, among others).

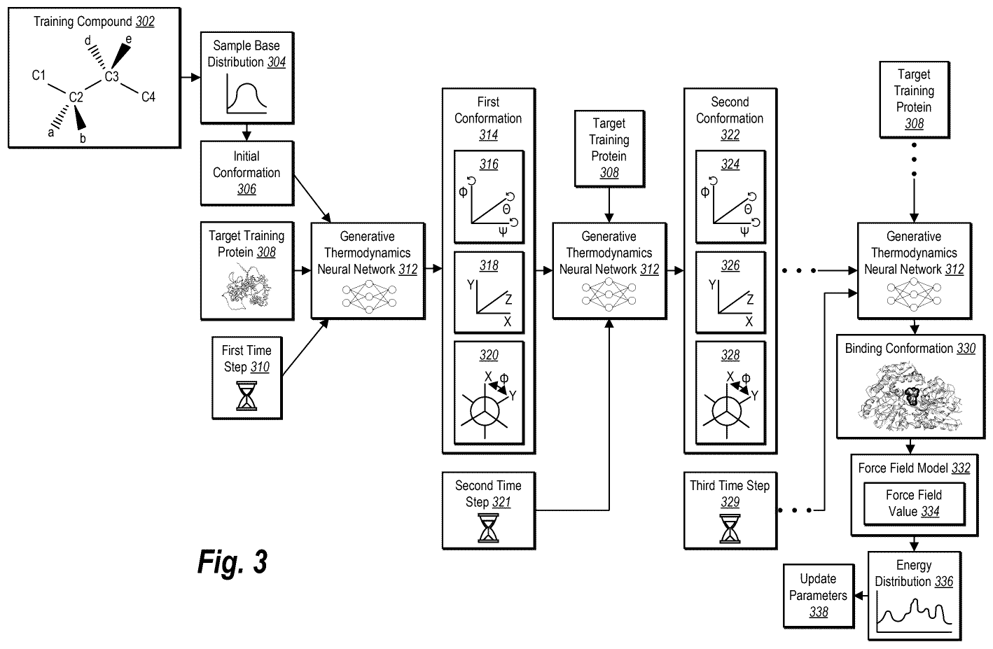

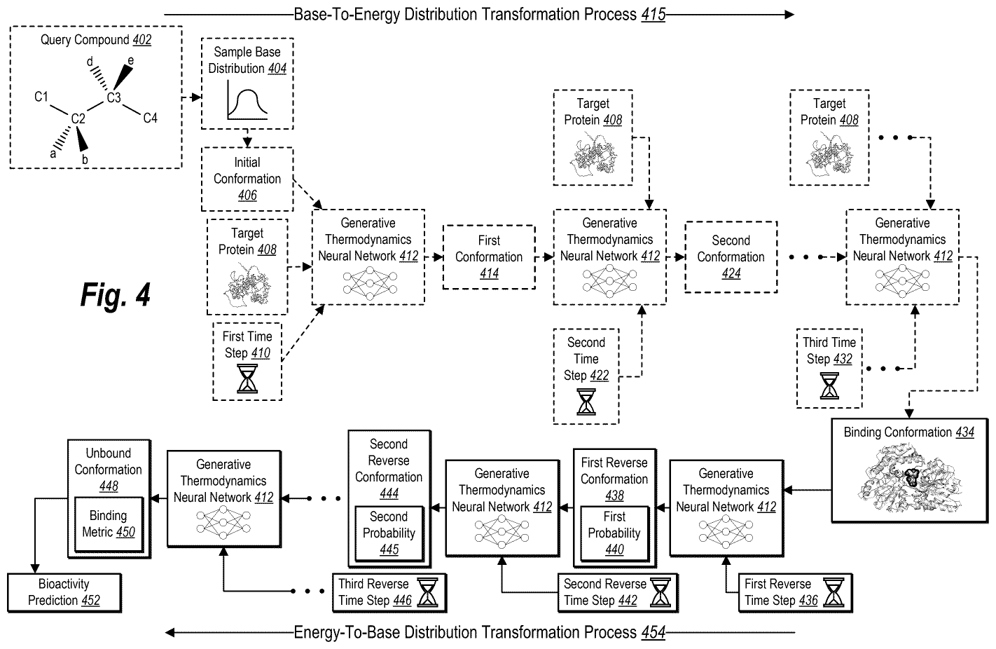

The thermodynamics binding system 100 can identify, access, or predict the binding conformation 110 in a variety of ways. For example, in some implementations, the thermodynamics binding system 100 receives the binding conformation 110 from a database or another computing device. In some implementations, the thermodynamics binding system 100 utilizes the generative thermodynamics neural network 108 to generate the binding conformation (e.g., in a base-to-energy distribution transformation process as described in greater detail below in relation to FIGS. 3 and 4).

Upon identifying the binding conformation 110, the thermodynamics binding system 100 can utilize the generative thermodynamics neural network 108 to map the binding conformation 110 to another conformation that corresponds to a base distribution 122. Indeed, as illustrated, the binding conformation 110 corresponds to the energy distribution 112 (e.g., energies for binding to a target protein) whereas the base distribution 122 reflects a base probability distribution (e.g., Gaussian distribution) of conformations for the query compound 102.

As illustrated, the thermodynamics binding system 100 can utilize an energy-to-base distribution transformation process 106 to determine a predicted series of conformations 114 for the query compound 102 from the binding conformation 110. As shown, the thermodynamics binding system 100 performs the energy-to-base distribution transformation process 106 by utilizing the generative thermodynamics neural network 108 to generate a predicted series of conformations 114. For instance, for a first time step from the binding conformation 110, the generative thermodynamics neural network 108 can predict a first conformation. In addition, for a second time step, the generative thermodynamics neural network can predict a second conformation from the first conformation. The thermodynamics binding system 100 can iteratively generate a series of conformations, resulting in a conformation reflecting the base distribution 122 of conformations of the query compound 102 (e.g., in an unbound state). Additionally, the thermodynamics binding system 100 can determine corresponding conformation probabilities between the binding conformation 110 and the unbound conformation of the query compound 102. The thermodynamics binding system 100 can utilize the conformation probabilities to determine the binding metric 116.

As illustrated, the thermodynamics binding system 100 can utilize the predicted series of conformations 114 to determine a binding metric 116. As used herein, the term “binding metric” refers to a measure of interaction/binding between a query compound and a target protein. In particular, a binding metric can indicate a measure of probability, likelihood, strength, or expected binding between the query compound 102 and the target protein 104. For instance, a binding metric can include a dissociation constant or K_{d} metric (e.g., a fraction of free ligand and free protein divided by the bound fraction of the concentration of the bound ligand protein).

Specifically, the thermodynamics binding system 100 can utilize the binding metric 116 to represent the binding interaction between the target protein and the query compound 102. In other words, the thermodynamics binding system 100 can use the conformation probabilities corresponding to the predicted series of conformations to determine a binding affinity between the initial conformation of the query compound and the target protein. Thus, by learning to map between a thermodynamics energy distribution (Boltzmann distribution of conformations) and a base distribution (e.g., Gaussian distribution of conformations), the thermodynamics binding system 100 can generate an accurate binding metric for a query compound relative to a target protein.

Although not illustrated, the thermodynamics binding system 100 can also train the generative thermodynamics neural network 108. In particular, the thermodynamics binding system 100 can train the generative thermodynamics neural network 108 by sampling a conformation from the base distribution 122 and utilizing a base-to-energy distribution transformation process to generate a binding conformation. The thermodynamics binding system 100 can determine an energy value (e.g., a force field value) corresponding to the binding conformation and utilize the energy value to determine a measure of loss to modify parameters of the generative thermodynamics neural network 108. In this manner, the generative thermodynamics neural network 108 can learn to map between the base distribution 122 and the energy distribution 112. Once trained, the thermodynamics binding system 100 can utilize the generative thermodynamics neural network 108 (as shown in FIG. 1) to perform the energy-to-base distribution transformation process 106 and generate binding metrics for query compounds and target proteins.

As illustrated in FIG. 1, the thermodynamics binding system 100 can also utilize the binding metric 116 to generate a bioactivity prediction 120, such as an ADMET prediction or a compound program analysis 118 for the query compound 102. For example, the thermodynamics binding system 100 can initiate the compound program analysis 118 by orchestrating a series of workflows to analyze the query compound 102 for future exploration. To illustrate, in some embodiments, the thermodynamics binding system 100 utilizes the compound program analysis 118 to generate a program rating for the query compound 102 for initiating a compound exploration program.

As mentioned briefly above, conventional systems suffer from a number of technical deficiencies with regard to implementing computing devices. For example, conventional systems require excessive computational resources and/or training data to predict binding affinities. In particular, some conventional systems utilize physics-based algorithms (such as molecular dynamics simulations or FEP), to model computational chemistry in predicting binding affinities. Although these methods can generate predicted affinities, they are computationally intensive and require significant computing resources to implement.

Other systems utilize supervised learning approaches to generate binding affinities. In particular, such systems generate binding predictions and compare these predictions with ground truth binding affinities to teach machine learning models to generate predicted binding affinities. However, these approaches rely on extensive training data sets that are time consuming and/or computationally expensive to generate. Thus, machine learning models are unable to generate accurate predictions without first undergoing the time and computational expense of generating or accessing a large corpus of reliable training data.

Conventional systems are also operationally inflexible. For instance, conventional systems are often unable to expand into new data domains or generate accurate predictions outside of particular data fields without first accessing or generating corresponding training data (e.g., measured binding affinities) for supervised learning. In other words, conventional systems are rigid in that scope of trained machine learning models are often tied to the underlying experimental domains on which they are trained.

As suggested by the foregoing discussion, the thermodynamics binding system 100 provides a variety of technical advantages relative to conventional systems. For example, the thermodynamics binding system 100 does not require binding affinity training data to train underlying models and generating accurate binding metrics. Indeed, the thermodynamics binding system 100 can train a generative thermodynamics neural network to map between an energy distribution and a base distribution through a series of conformations. Specifically, instead of ground truth binding affinities, the thermodynamics binding system 100 can utilize a measure of energy associated with predicted binding conformations to train the generative thermodynamics neural network. Because the thermodynamics binding system 100 can predict binding metrics according to probabilities corresponding to a predicted series of conformations in mapping from an energy distribution to a base distribution, the thermodynamics binding system 100 does not require ground truth binding affinity training data. Accordingly, the thermodynamics binding system 100 is less computationally expensive and more efficient than traditional physics-based or machine learning based systems.

In addition to the improvements regarding computational resources, in some embodiments, the thermodynamics binding system 100 improves upon operational flexibility. For example, the thermodynamics binding system 100 does not need to rely on experimental/measured binding measures to train a machine learning model. Accordingly, the thermodynamics binding system 100 can expand to a variety of domains, even where similar compounds have not been experimentally analyzed in the past. Indeed, by utilizing energy measures to learn to map to the Boltzmann distribution of binding conformations, the thermodynamics binding system 100 can accurately predict binding metrics across untested data domains.

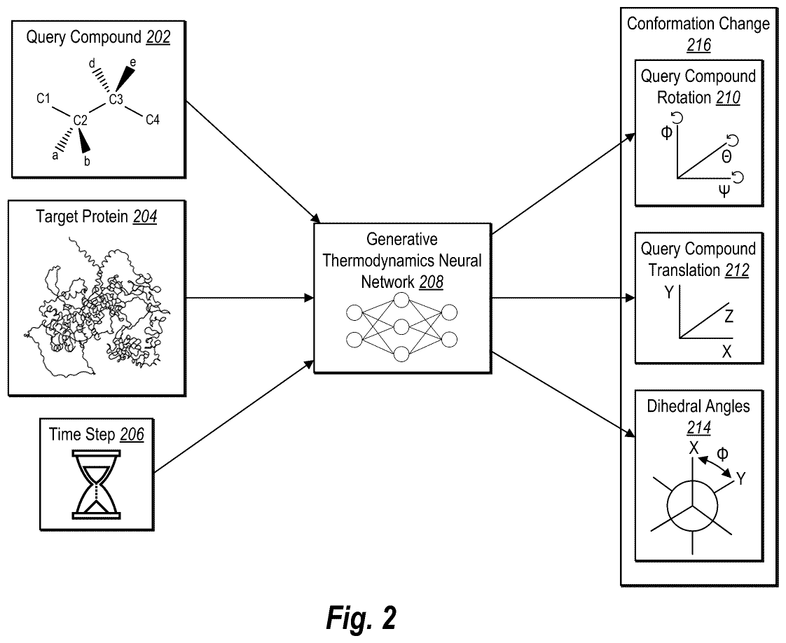

As just mentioned, in one or more implementations the thermodynamics binding system 100 can utilize a generative thermodynamics neural network to generate conformations for a query compound. For example, FIG. 2 illustrates the thermodynamics binding system 100 utilizing a generative thermodynamics neural network to generate a conformation change for a query compound.

As used herein, the term “generative thermodynamics neural network” (or generative thermodynamics machine learning model) refers to a machine learning model trained to generate conformations for query compounds based on energy levels (e.g., thermodynamics). As used herein, the term “machine learning model” includes a computer algorithm or a collection of computer algorithms that can be trained and/or tuned based on inputs to approximate unknown functions. For example, a machine learning model can include a computer algorithm with branches, weights, or parameters that are changed based on training data to improve for a particular task. Thus, a machine learning model can utilize one or more learning techniques (e.g., supervised or unsupervised learning) to improve in accuracy and/or effectiveness. Example machine learning models include various types of decision trees (e.g., gradient boost models), support vector machines, Bayesian networks, random forest models, or neural networks (e.g., deep neural networks, generative adversarial neural networks, convolutional neural networks, recurrent neural networks, or diffusion neural networks). Similarly, as used herein, a neural network refers to a machine learning model of interconnected nodes (or neurons) organized into layers. A neural network can include parameters or weights between neurons that are adjusted during training to minimize the error (or measure of loss) in generating predictions.

Thus, a generative thermodynamics neural network includes a neural network trained to generate conformations by modeling thermodynamic energy levels in binding a compound to a protein. Specifically, a generative thermodynamics neural network includes a neural network trained to map a compound between a base distribution (e.g., a Gaussian distribution) to an energy distribution (e.g., a Boltzmann distribution or other distribution of energy). Thus, a generative thermodynamics neural network can predict conformations (e.g., from an unbound state to a bound state or from a bound state to an unbound state). In one or more implementations, the generative thermodynamics neural network is implemented as a graph neural network. A graph neural network refers to a type of neural network designed to process data represented as graphs, where nodes represent entities and edges represent relationships between them.

As illustrated in FIG. 2, the thermodynamics binding system 100 can provide a query compound 202, a target protein 204, and a time step 206 to a generative thermodynamics neural network 208. The query compound 202 can be the query compound 102 of FIG. 1. Indeed, the thermodynamics binding system 100 can sample an initial conformation of the query compound from a base distribution, and provide the initial conformation to the generative thermodynamics neural network. Further, the query compound 202 can be a graph representation of a compound. For example, the query compound 202 can include nodes corresponding to atoms of the compound, and edges corresponding to bonds between the molecules of the compound. In some embodiments, the edges can represent non-covalent interactions between the molecules of the compound.

Moreover, the target protein 204 can be a biological target of interest for the thermodynamics binding system 100. For example, the target protein 204 can be related to a particular biological process (e.g., a protein predicted to interact with a compound within a cell). The thermodynamics binding system 100 can utilize the generative thermodynamics neural network to simulate one or more binding interactions between the query compound 202 and the target protein over time.

As used herein, the term “time step” refers to a stage, step, time or element in a series of stages, steps, times, or elements. For example, a time step can include a single conformation step in a series of conformations (e.g., between a binding conformation and another conformation) For example, a first timestep can refer to a first stage of conforming a query compound (from an initial conformation) to align to a target protein Thus, a first time step can correlate to a first predicted conformation change for the query compound in a multi-stage conformation process to bind the query compound to the target protein.

As illustrated, the thermodynamics binding system 100 can utilize the generative thermodynamics neural network 208 to analyze the query compound 202 and the target protein 204 for the time step 206. For instance, in some implementations, the generative thermodynamics neural network 208 can be an equivariant graph neural network or an SE (3) transformer. Indeed, the thermodynamics binding system 100 can utilize the generative thermodynamics neural network to perform an equivariant convolution on the query compound 202. The thermodynamics binding system 100 can utilize the results of the equivariant convolution to update the graph representation (e.g., the thermodynamics binding system 100 updates the nodes and edges of the graph representation) of the query compound 202. The thermodynamics binding system 100 utilizes the updated graph representation of the query compound 202 to predict the conformation change 216.

Specifically, the thermodynamics binding system 100 can utilize the updated graph representation to predict a query compound rotation 210, a query compound translation 212, and dihedral angles 214 of the query compound (e.g., a first set of modified dihedral angles of the query compound). For example, the thermodynamics binding system 100 can aggregate the node features of the query compound 202 into a single vector and input the vector into a layer of the generative thermodynamics neural network (e.g., an equivariant layer). Responsive to receiving the vector input, the thermodynamics binding system 100 can generate a first vector describing the query compound rotation 210 and a second vector describing the query compound translation 212.

Moreover, the thermodynamics binding system 100 can combine the updated node features for each of the atoms in each dihedral angle of the query compound 202 and inputs the dihedral angle node features to the generative thermodynamics neural network. For example, the thermodynamics binding system 100 can concatenate the updated node features. In addition, the thermodynamics binding system 100 can identify dihedral angles according to groups of up to four interconnected nodes of the graph representation. In some embodiments, the thermodynamics binding system 100 can input the dihedral angle node features into a multi-layer perceptron (MLP). Responsive to receiving the dihedral angle node features, the thermodynamics binding system 100 outputs a new dihedral angle for each dihedral angle of the query compound.

The thermodynamics binding system 100 can combine the query compound rotation 210, the query compound translation 212, and the dihedral angles 214 to generate the conformation change 216. In some embodiments, the thermodynamics binding system 100 can iteratively determine conformation changes. For example, the thermodynamics binding system 100 can utilize the generative thermodynamics neural network to analyze a first conformation (resulting from the conformation change), the target protein, and a second time step to determine a second confirmation change. More information regarding the thermodynamics binding system 100 iteratively generating conformation changes will be provided below with regards to FIGS. 3 and 4.

As mentioned above, the thermodynamics binding system 100 can train a generative thermodynamics neural network to utilize a base-to-energy distribution transformation process to learn a binding conformation for a training compound. FIG. 3 depicts the thermodynamics binding system 100 training a generative thermodynamics neural network to learn to utilize a base-to-energy distribution transformation process to determine a binding conformation from a training compound.

As illustrated in FIG. 3, the thermodynamics binding system 100 can receive a training compound 302. The training compound 302 can be a representation of a chemical compound, such as a ligand. For example, the thermodynamics binding system 100 can receive the training compound in the form of a chemical formula. Specifically, the thermodynamics binding system 100 can receive the training compound 302 and a target training protein 308 as part of a training binding query. Additionally, the training binding query can include a prompt requesting more information about a binding interaction between the training compound 302 and the target training protein 308.

After receiving the training compound 302, the thermodynamics binding system 100 can perform an act 304 of sampling a base distribution of the training compound 302. Specifically, the thermodynamics binding system 100 can perform the act 304 of sampling the initial conformation 306 of the training compound 302 from a base distribution. As used herein, the term “base distribution” refers to a baseline or estimated distribution of conformations of a compound. For instance, a base distribution can include a probability distribution (e.g., a Gaussian or other modeled distribution) of unbound conformations for a compound. Specifically, the thermodynamics binding system 100 can determine a base distribution (e.g., a known distribution such as a Gaussian distribution) of conformations of the training compound 302 and sample an initial conformation 306 from the base distribution. For example, the thermodynamics binding system 100 can sample an initial conformation representative of a particular spatial arrangement (e.g., the same chemical formula as the training compound 302 but differing in spatial arrangement such as stereochemistry and/or organization of molecules/functional groups) of the training compound 302. In one more implementations, the thermodynamics binding system 100 generates features (e.g., three-dimensional coordinates or other features) of the initial conformation to analyze utilizing the generative thermodynamics neural network 312.

As shown, the thermodynamics binding system 100 can utilize the generative thermodynamics neural network 312 to analyze the initial conformation 306, the target training protein 308 for a first time step 310. The thermodynamics binding system 100 can utilize the first time step 310 to model a first stage for modifying the training compound 302 to bind with the target training protein 308.

As shown, the thermodynamics binding system 100 utilizes the generative thermodynamics neural network 312 to perform a base-to-energy distribution transformation process to generate a first conformation 314 of the training compound 302 (e.g., a first conformation in the binding interaction between the training compound 302 and the target training protein 308). As used herein, the term “base-to-energy distribution transformation process” refers to mapping a compound from a base distribution to an energy distribution. In particular, a base-to-energy distribution transformation process can include generating a series of conformations of a compound from an initial/unbound conformation (e.g., a shape or arrangement of an unbound compound sampled from a base distribution) to a binding conformation (e.g., a shape or arrangement of a compound that reflects thermodynamic energy of the compound binding to a protein). Thus, a base-to-energy distribution transformation process includes modeling transformation of a compound from an unbound state corresponding to a base distribution of conformations to a bound state corresponding to a Boltzmann distribution of conformations.

Moreover, as used herein, the term “energy distribution” refers to a distribution of conformations of a compound according to an energy (e.g., a thermodynamic energy) associated with the conformations. In particular, an energy distribution includes a probability distribution of energy among particles in a system at equilibrium. Thus, an energy distribution includes a probability distribution of conformations associated with binding a compound to a protein. In some implementations, an energy distribution includes a Boltzmann distribution.

As illustrated in FIG. 3, the thermodynamics binding system 100 can generate the first conformation 314 by generating various features, such as rotation, translation, and modified dihedral angles. Specifically, the thermodynamics binding system 100 can generate a first training compound rotation 316 (e.g., a first rotation of the training compound) in the first conformation 314. Indeed, the thermodynamics binding system 100 can utilize the first training compound rotation 316 to represent rotations of the training compound 302 during a first step of conforming the training compound 302 to bind with the target training protein 308. For example, the thermodynamics binding system 100 can utilize the first training compound rotation 316 to quantify rotation of the initial conformation 306 of the training compound 302 about one or more axes.

Additionally, as illustrated in FIG. 3, the thermodynamics binding system 100 can generate a first training compound translation 318 (e.g., a first translation of the training compound) in the first conformation 314. Specifically, the thermodynamics binding system 100 can utilize the first training compound translation 318 to represent translation of the training compound during the first step of the binding interaction between the training compound 302 and the target training protein 308. For example, the thermodynamics binding system 100 can utilize the first training compound translation 318 to quantify translation of the initial conformation 306 of the training compound 302 along one or more axes.

Moreover, as shown in FIG. 3, the thermodynamics binding system 100 can generate first dihedral angles 320 (e.g., a first set of modified dihedral angles) in the first conformation 314. Specifically, the thermodynamics binding system 100 can utilize the first dihedral angles 320 to represent rotations of various molecules of the training compound 302 about a bond between molecules of the training compound in the first step of the binding interaction between the training compound 302 and the target training protein 308. In other words, the thermodynamics binding system 100 can utilize the first dihedral angles 320 to quantify intramolecular rotations of the initial conformation 306 of the training compound 302. The thermodynamics binding system 100 can include one or more dihedral angles in the first dihedral angles 320. For example, in the first step of the binding interaction, one or more molecules (or groups of molecules, i.e. functional groups) of the initial conformation 306 of the training compound 302 can rotate around one or more bonds of the training compound 302. The thermodynamics binding system 100 can determine a dihedral angle for each rotation of a molecule around a bond and can include each of the dihedral angles in the first dihedral angles 320.

Indeed, the thermodynamics binding system 100 can utilize the first training compound rotation 316, the first training compound translation 318, and the first dihedral angles 320 to quantify conformational changes to the initial conformation 306 of the training compound 302 during the first step of the binding interaction between the training compound 302 and the target training protein 308. The thermodynamics binding system 100 can utilize these conformational changes to represent the first conformation 314 of the training compound 302 from the initial conformation 306.

As illustrated in FIG. 3, the thermodynamics binding system 100 can also utilize the generative thermodynamics neural network 312 to analyze the first conformation 314, the target training protein 308, and a second time step 321. The thermodynamics binding system 100 can utilize the second time step 321 to indicate a next stage in the binding interaction between the training compound 302 and the target training protein 308.

The thermodynamics binding system 100 can cause the generative thermodynamics neural network 312 to perform an iteration of the base-to-energy distribution transformation process to determine a second conformation 322 of the training compound 302. The thermodynamics binding system 100 can represent the output of the generative thermodynamics neural network 312 as the second conformation 322.

Indeed, similar to how the thermodynamics binding system 100 can utilize the first conformation 314 to represent conformational changes to the initial conformation 306 of the training compound 302, the thermodynamics binding system 100 can utilize the second conformation 322 to represent conformational changes to the first conformation 314 of the training compound 302. The thermodynamics binding system 100 can utilize the second training compound rotation 324 to represent rotation of the first conformation 314 of the training compound 302 about one or more axes. Additionally, the thermodynamics binding system 100 can utilize the second training compound translation 326 to represent rotation of the first conformation 314 of the training compound around one or more axes. Moreover, the thermodynamics binding system 100 can utilize the second dihedral angles 328 to represent intramolecular rotations of one or more molecules around one or more bonds of the first conformation 314 of the training compound 302.

As illustrated in FIG. 3, the thermodynamics binding system 100 can input the second conformation 322, the target training protein 308, and a third time step 329 into the generative thermodynamics neural network 312. Indeed, the thermodynamics binding system 100 can iteratively analyze conformations of the training compound 302 along with corresponding time steps and the target training protein 308 utilizing the generative thermodynamics neural network 312. The thermodynamics binding system 100 can utilize the generative thermodynamics neural network 312 to iteratively generate conformations to perform the base-to-energy distribution transformation process and model conformations of the training compound 302 corresponding to phases of the binding interaction between the training compound 302 and the target training protein 308.

After iteratively generating conformations to perform the base-to-energy distribution transformation process, the thermodynamics binding system 100 can determine a binding conformation 330 of the training compound 302. Specifically, the thermodynamics binding system 100 can utilize the binding conformation 330 to represent a conformation of the training compound that binds to the target training protein 308.

As illustrated, the thermodynamics binding system 100 can utilize a force field model 332 to analyze the binding conformation 330. As used herein, the term “force field model” refers to a computer-implemented algorithm for generating or predicting a measure of energy corresponding to a compound. In particular, a force field model can include a computer-implemented algorithm that models potential energy of a molecule based on the positions of the atoms of the molecule and their interactions (e.g., how the atoms are bound).

Indeed, the thermodynamics binding system 100 can utilize the force field model 332 to predict a measure of energy of the binding conformation 330. Specifically, the thermodynamics binding system 100 can utilize the force field model 332 to simulate molecular dynamics of the binding conformation. Additionally, the thermodynamics binding system 100 can utilize the force field model 332 to estimate various energetic properties of the binding conformation 330, such as bond association energies, interaction energies (e.g., energy associated with non0bonded interactions, such as van der Waals forces and electrostatic interactions), and reaction energies (e.g., energy changes during chemical reactions). Additionally, the thermodynamics binding system 100 can utilize the force field model 332 to estimate a potential energy of the binding conformation 330.

Specifically, the thermodynamics binding system 100 can utilize the force field model 332 to estimate the potential energy of the binding conformation 330 by analyzing various features of the binding conformation 330. Specifically, the thermodynamics binding system 100 can utilize the force field model 332 to determine a potential energy from various aspects of the binding conformation 330, including: bond stretching (e.g., the energy associated with the stretching and compressing of bonds between two atoms of the binding conformation 330), angle bending (e.g., the energy required to bend bond angles of the binding conformation 330 away from their equilibrium positions), dihedral angles (e.g., torsional interactions, or the energy associated with rotation around bonds of the binding conformation 330), and non-bonded interactions (e.g., forces from non-bonded interactions, including attractive and repulsive forces between atoms of the binding conformation 330).

The thermodynamics binding system 100 can utilize a value from the force field model 332, such as the potential energy of the binding conformation 330 discussed above, as a force field value 334. Specifically, the thermodynamics binding system 100 can use the force field value 334 to represent a level of stability of the binding conformation 330, where a lower force field value represents a higher level of stability of the binding conformation 330. Thus, a force field model can include a model for generating a potential energy of a compound in a binding conformation relative to a target protein. For example, a force field model can include APLS-All-Atom (OPLS-AA), OPLS3, CHARMM General force field (CGenFF), General Amber Force Field (GAFF), Merck Molecular Force Field (MMFF), or GROMOS.

As illustrated, the thermodynamics binding system 100 can utilize the force field model 332 and corresponding force field value 334 (e.g., an energy value) to perform an act 338 of updating parameters of the generative thermodynamics neural network 312. The thermodynamics binding system 100 can update parameters/train the generative thermodynamics neural network 312 utilizing a variety of approaches. For example, in some implementations, the thermodynamics binding system 100 utilizes the force field value 334 as a measure of loss and back-propagate to reduce the measure of loss. In this manner, the thermodynamics binding system 100 can model the energy distribution by learning parameters the minimize the measure of energy associated with the molecular system.

In some implementations, the thermodynamics binding system 100 compares a predicted probability or probability distribution with the energy distribution 336 (e.g., the Boltzmann distribution). In particular, the thermodynamics binding system 100 can convert the force field value 334 to a probability. For example, the thermodynamics binding system 100 can utilize a Boltzmann model (e.g., a computer model for implementing the Boltzmann equation to determine a probability of a particle having a particular energy at a particular temperature) to generate a probability for the force field value 334.

In addition, the thermodynamics binding system 100 can determine a predicted probability utilizing the generative thermodynamics neural network 312. Indeed, one or more layers of the generative thermodynamics neural network 312 generates probabilities of various conformations that are utilized to select the binding conformation 330. The thermodynamics binding system 100 can compare a predicted probability for the binding conformation 330 generated by the generative thermodynamics neural network 312 with the probability generated from the force field value 334. In particular, the thermodynamics binding system 100 can determine a measure of loss by comparing these probabilities. Moreover, the thermodynamics binding system 100 can update parameters of the generative thermodynamics neural network 312 to reduce the measure of loss (e.g., utilizing gradient descent and back propagation).

In some implementations, the thermodynamics binding system 100 can compare predicted probability distributions predicted by the generative thermodynamics neural network 312 with probability distributions determined utilizing the force field model 332 to perform the act 338 of updating parameters. As just mentioned, one or more layers of the generative thermodynamics neural network 312 can generate a predicted probability distribution for conformations (e.g., the probability distribution utilized to sample/select a binding conformation). Similarly, the thermodynamics binding system 100 can convert, utilizing the Boltzmann model, various force field values for binding conformations to an energy distribution 336 The thermodynamics binding system 100 can compare the predicted probability distribution (from the generative thermodynamics neural network 312) with the energy distribution 336 generated by the force field model 332. For instance, the thermodynamics binding system 100 can utilize an inverse Kullback-Leibler loss function to compare the predicted probability distribution across binding conformations and the energy distribution 336 across binding conformations to generate a measure of loss. The thermodynamics binding system 100 can then utilizes the measure of loss to modify parameters of the generative thermodynamics neural network 312. Accordingly, the thermodynamics binding system 100 can teach the generative thermodynamics neural network 312 to learn to map the sample base distribution to the energy distribution 336 (e.g., the Boltzmann distribution).

Although not illustrated in FIG. 3, in some embodiments, the thermodynamics binding system 100 can also determine rotatable bonds in the training protein at each time step (e.g., the first time step, the second time step, the third time step, etc.). Accordingly, the thermodynamics binding system 100 can determine a conformation for the training compound and the target training protein at each time step. By determining conformations for the training compound and the target training protein, the thermodynamics binding system 100 can train the generative thermodynamics neural network to produce more accurate binding conformations, thereby determining more accurate energy values.

As mentioned above, the thermodynamics binding system 100 can utilize a generative thermodynamics neural network to determine a binding metric for a query compound. For example, FIG. 4 illustrates the thermodynamics binding system 100 receiving a query compound, generating a binding conformation for the query compound utilizing a base-to-energy distribution transformation process 415, and determining a binding metric for the binding conformation utilizing an energy-to-base distribution transformation process 454.

As illustrated in FIG. 4, the thermodynamics binding system 100 can receive a query compound 402. Specifically, the thermodynamics binding system 100 can receive a binding query for the query compound 402 and the target protein 408. The binding query can also include a query or request for the thermodynamics binding system 100 to provide more information about the binding interaction between the query compound 402 and the target protein 408.

The thermodynamics binding system 100 can perform an act 404 to sample a base distribution of the query compound 402 to obtain an initial conformation 406 of the query compound 402. The base distribution can be a known distribution, such as a Gaussian distribution, of conformations (e.g., spatial conformations that differ in aspects such as stereochemistry and connectivity) of the query compound 402.

As illustrated, the thermodynamics binding system 100 can input the initial conformation 406 of the query compound 402, the target protein 408, and a first time step 410 to a generative thermodynamics neural network 412. The generative thermodynamics neural network 412 can be a generative thermodynamics neural network that has undergone the training process described previously with respect to FIG. 3. Responsive to receiving the initial conformation 406, the target protein 408, and the first time step 410, the thermodynamics binding system 100 can cause the generative thermodynamics neural network 412 to determine a first conformation 414 of the query compound. The thermodynamics binding system 100 can utilize the first conformation 414 to represent a change to the initial conformation in a step of the binding interaction between the query compound 402 and the target protein 408.

Indeed, the thermodynamics binding system 100 can determine various spatial changes from the initial conformation 406 in the first conformation 414. For example, as a part of the first conformation, the thermodynamics binding system 100 can include a first rotation of the query compound, which the thermodynamics binding system 100 can use to represent rotation of the initial conformation about one or more axes. Additionally, the thermodynamics binding system 100 can include a first translation of the query compound as part of the first conformation 314. Specifically, the thermodynamics binding system 100 can utilize the first translation of the query compound to represent translation of the initial conformation 406 along one or more axes. Moreover, the thermodynamics binding system 100 can include a first set of modified dihedral angles of the query compound in the first conformation 414. Specifically, the thermodynamics binding system 100 can utilize the first set of modified dihedral angles of the query compound to represent changes to dihedral angles of the initial conformation (e.g., intramolecular rotations of atoms of the initial conformation 406 about a bond of the query compound 402).

As illustrated in FIG. 4, the thermodynamics binding system 100 can input the target protein 408, the first conformation 414, and a second time step 422 into the generative thermodynamics neural network 412. The thermodynamics binding system 100 can cause the generative thermodynamics neural network 412 to generate a second conformation 424 of the query compound. Indeed, the thermodynamics binding system 100 can include a second rotation of the query compound in the second conformation 424. The thermodynamics binding system 100 can utilize the second rotation of the query compound to represent one or more rotations of the query compound 402 (e.g., the initial conformation 406 of the query compound 402) about one or more axes relative to the first conformation 414. Additionally, the thermodynamics binding system 100 can include a second translation of the query compound in the second conformation 424. The thermodynamics binding system 100 can utilize the second translation of the query compound to represent translation of the query compound 402 (e.g., the initial conformation 406 of the query compound 402) along one or more axes relative to the first conformation 414. Moreover, the thermodynamics binding system 100 can include a second set of modified dihedral angles of the query compound in the second conformation 424. Specifically, the thermodynamics binding system 100 can utilize the second set of modified dihedral angles of the query compound to represent changes in the dihedral angles of the query compound 402 (e.g., the first conformation 414 of the query compound) relative to the first conformation 414.

In some embodiments, the thermodynamics binding system 100 can iteratively input conformations, time steps corresponding to the conformations, and the target protein 408 into the generative thermodynamics neural network to determine subsequent conformations of the query compound. For example, the thermodynamics binding system 100 can input the second conformation 424, the third time step 432, and the target protein 408 into the generative thermodynamics neural network to determine a third conformation. The thermodynamics binding system 100 can include a third rotation of the query compound, a third translation of the query compound, and a third set of modified dihedral angles of the query compound.

In this manner, the thermodynamics binding system 100 can determine a binding conformation 434 for the query compound 402 relative to the target protein 408. Moreover, in some embodiments, the thermodynamics binding system 100 can generate an ensemble of binding conformations (e.g., a plurality of binding conformations) for the query compound 402 and the target protein 408. That is to say, the thermodynamics binding system 100 can determine multiple binding conformations from the query compound 402 and the target protein. Indeed, the thermodynamics binding system 100 can utilize the base-to-energy distribution transformation process 415 to generate the binding conformation 434 from the initial conformation 406. Notably, once trained, the generative thermodynamics neural network 412 selects the binding conformation 434 by modeling the energy distribution (e.g., Boltzmann distribution) of the query compound 402 relative to the target protein. Thus, the generative thermodynamics neural network 412 maps the base distribution to the energy distribution for a binding of the query compound 402 to the target protein 408.

Notably, as denoted by the dashed lines in FIG. 4, the thermodynamics binding system 100 can generate the binding conformation 434 utilizing the base-to-energy distribution transformation process 415 or access the binding conformation 434 through an alternative approach. Indeed, in some implementations, the thermodynamics binding system 100 identifies or receives a binding conformation (e.g., from a third-party server or with the query compound 402). The thermodynamics binding system 100 can utilize the binding conformation 434 received from an alternative source or the thermodynamics binding system 100 can generate the binding conformation 434 as illustrated.

In some implementations, the thermodynamics binding system 100 can determine how many conformations, if any, to generate for a query compound 402. For example, the thermodynamics binding system 100 can repeat the base-to-energy distribution transformation process 415 and select a plurality of different binding conformations. Similarly, the thermodynamics binding system 100 can receive a plurality of different binding conformations from other sources. In one or more implementations, the thermodynamics binding system 100 models all binding states (i.e., all binding conformations) of the query compound relative to a target protein.

As shown in FIG. 4, regardless of the source of the binding conformation 434, the thermodynamics binding system 100 can utilize the energy-to-base distribution transformation process 454 to determine a binding metric 450 for the binding conformation 434. As used herein, the term “energy-to-base distribution transformation process” refers to mapping a compound from an energy distribution to a base distribution. In particular, an energy-to-base distribution transformation process can include generating a series of conformations of a compound from a binding conformation (e.g., a shape or arrangement of a compound that reflects thermodynamic energy of the compound binding to a protein) to an unbound conformation (e.g., a shape or arrangement of an unbound compound sampled from a base distribution). Thus, a base-to-energy distribution transformation process includes modeling transformation of a compound from a bound state corresponding to a Boltzmann distribution of conformations to an unbound state corresponding to a base distribution of conformations.

Moreover, as used herein, the term “predicted series of conformations” refers to a sequence of conformations from one state of a compound to another. For example, a predicted series of conformations can include a sequence of conformations from a binding conformation to an unbound conformation (or vice versa).

As illustrated in FIG. 4, the thermodynamics binding system 100 can input the binding conformation 434, and a first reverse time step 436 into the generative thermodynamics neural network 412. Specifically, the binding conformation 434 can be a conformation of the query compound 402 that is bound to the target protein 408. As used herein, variations of the phrase “reverse time step” (such as first reverse time step, second reverse time step, third reverse time step etc.) refer to a stage, step, or element within the energy-to-base distribution transformation process. The phrase “reverse time step” does not imply that time is moving backwards, but instead refer to the thermodynamics binding system 100 utilizing the energy-to-base distribution transformation process 454 to transform the binding conformation 434 to an unbound conformation 448 of the query compound. Accordingly, similarly to how time steps in the energy-to-base distribution transformation process correlate with conformations of the query compound during a binding interaction between the query compound (e.g., a first conformation of the query compound), reverse time steps correlate with conformations of the query compound during a dissociation reaction of the binding conformation (e.g., the query compound dissociating or becoming unbonded from the target protein).

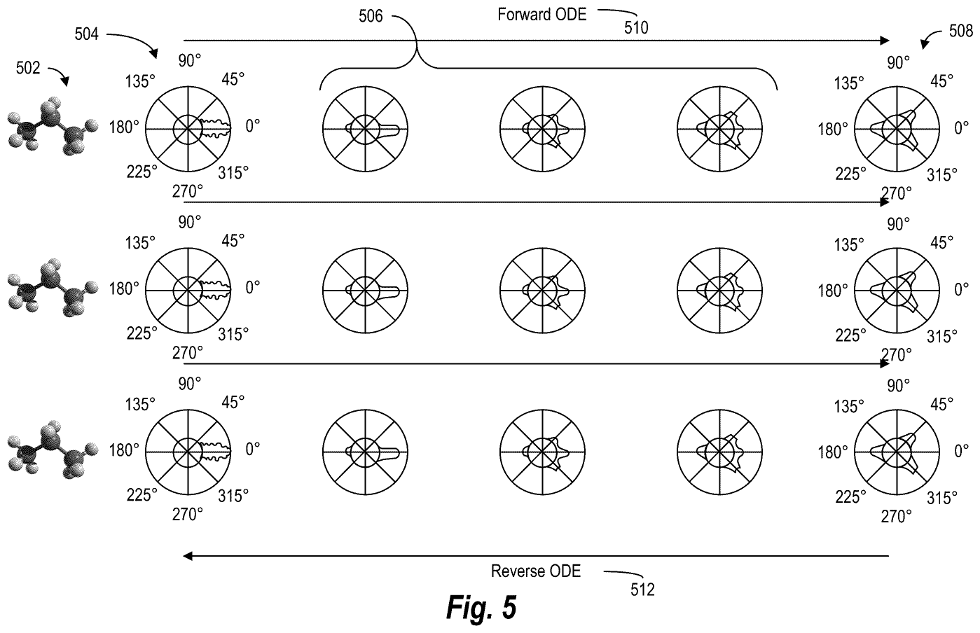

Responsive to inputting the binding conformation 434 and the first reverse time step 436 into the generative thermodynamics neural network 412, the thermodynamics binding system 100 can cause the generative thermodynamics neural network 412 to generate a first reverse conformation 438 for the query compound. More information regarding the energy-to-base distribution transformation process will be provided below with regard to FIG. 5.

As illustrated, the thermodynamics binding system 100 can utilize the first reverse conformation 438 to represent a conformation of the query compound in a dissociation reaction between the query compound 402 and the target protein. Specifically, the thermodynamics binding system 100 can determine a first probability 440 associated with the first reverse conformation 438. For instance, the generative thermodynamics neural network 412 can generate a conformation and corresponding probability associated with the conformation. Indeed, the thermodynamics binding system 100 can utilize the first probability 440 to represent a probability of the query compound 402 changing conformations from the binding conformation 434 to the first reverse conformation 438.

As shown in FIG. 4, the thermodynamics binding system 100 can input the first reverse conformation 438 and the second reverse time step 442 into the generative thermodynamics neural network 412. Responsive to receiving these inputs, the thermodynamics binding system 100 can cause the generative thermodynamics neural network 412 to utilize the energy-to-base distribution transformation process 454 to determine a second reverse conformation 444 and a second probability 445. Specifically, the thermodynamics binding system 100 can utilize the second probability 445 to represent a likelihood that the query compound will change conformations from the first reverse conformation to the second reverse conformation 444. Additionally, the thermodynamics binding system 100 can input the second reverse conformation 444 and a third reverse time step 446 into the generative thermodynamics neural network 412 to cause the generative thermodynamics neural network 412 to generate an additional reverse conformation.

Indeed, as illustrated in FIG. 4, the thermodynamics binding system 100 can iteratively input reverse conformations and reverse time steps into the generative thermodynamics neural network 412 to generate conformations and corresponding probabilities for the query compound 402. The thermodynamics binding system 100 can generate the unbound conformation 448 for the query compound 402. The unbound conformation 448 can be a conformation of the query compound 402 corresponding to the base distribution of the query compound. Indeed, the generative thermodynamics neural network can learn to transform between an energy distribution and a base distribution. Thus, the generative thermodynamics neural network 412 can internally model the base distribution in generating the unbound conformation 448.

Additionally, the thermodynamics binding system 100 can combine (e.g., sum, multiply, etc.) the probabilities associated with the reverse conformations (e.g., the first probability 440, the second probability 445, etc.) to determine the binding metric 450. Specifically, the thermodynamics binding system 100 can determine the binding metric 450 as being inversely proportional to the sum of probabilities of dissociation (e.g., the first probability 440, the second probability 445, etc.). That is to say, that a high sum of probabilities of dissociation correlates to a low binding metric 450, whereas a low sum of probabilities of dissociation correlates to a high binding metric 450.

Although FIG. 4 illustrates generating the binding metric 450 based on a single binding conformation, in some implementations, the thermodynamics binding system 100 performs the energy-to-base distribution transformation process 454 for an ensemble of binding conformations, determines probabilities from this plurality of binding conformations and generates the binding metric by combining the resulting probabilities. In some implementations, the thermodynamics binding system 100 can perform the energy-to-base distribution transformation process 454 for all binding conformations of a compound relative to a protein, and combine corresponding conformation probabilities for the energy-to-base distribution transformation process 454 across the binding conformations to determine the binding metric 450.

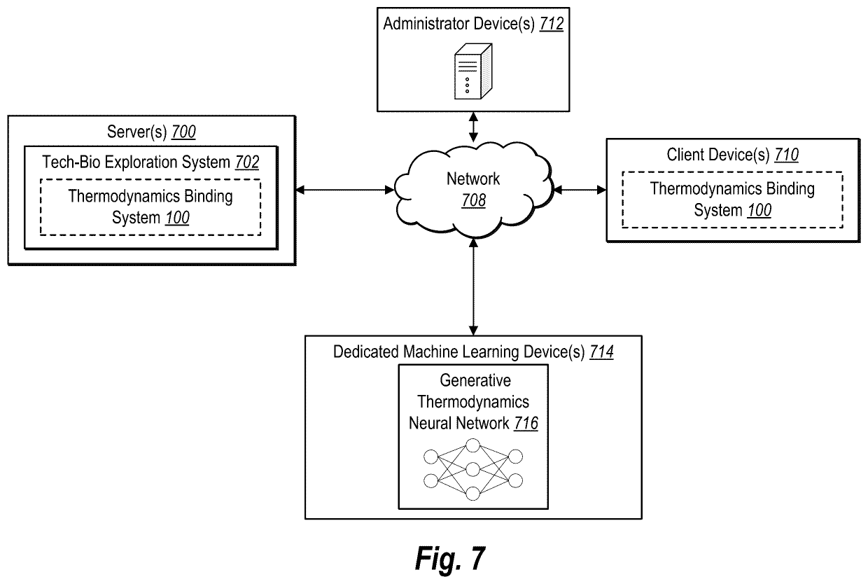

As shown in FIG. 4, the thermodynamics binding system 100 can utilize the binding metric 450 to generate the bioactivity prediction 452. In some embodiments, the bioactivity prediction 452 can be the compound program analysis 118 of FIG. 1. Additionally or alternatively, the bioactivity prediction 452 can be a transcriptomic prediction for the query compound. More information regarding the bioactivity prediction can be found below with regard to FIG. 7.

As mentioned above, the thermodynamics binding system 100 can utilize a base-to-energy distribution transformation process to generate a binding conformation for a query compound. Additionally, the thermodynamics binding system 100 can utilize an energy-to-base distribution transformation process to generate a binding metric for the query compound. FIG. 5 depicts the thermodynamics binding system 100 utilizing the base-to-energy distribution transformation process and the energy-to-base distribution transformation process to determine a binding conformation and a binding metric for a plurality of query compounds. In particular, FIG. 5 illustrates the thermodynamics binding system 100 utilizing the base-to-energy distribution transformation process to generate a series of conformations for each of a plurality of query compounds resulting in a binding conformation for each of the plurality of query compounds, and then utilizing the energy-to-base distribution transformation process to generate a predicted series of conformations and corresponding conformation probabilities between the binding conformation and an unbound conformation of the query compound.

As illustrated, FIG. 5 depicts a first compound 502 (e.g., a query compound or a training compound) an initial conformation 504 of the first compound, a predicted series of conformations 506 corresponding to a binding interaction between the initial conformation 504 and a target protein, and a binding conformation 508 representative of a final conformation of the first compound 502 in the binding interaction between the first compound 502 and the target protein. The thermodynamics binding system 100 can sample the initial conformation 504 from a known distribution of the first compound, such as a Gaussian distribution or a wrapped Gaussian Distribution. The thermodynamics binding system 100 can determine the binding conformation 508 utilizing a forward ordinary differential equation 510 (reproduced below). The thermodynamics binding system 100 can determine the binding metric using a reverse ordinary differential equation 512.

The thermodynamics binding system 100 utilizes various forms of the following base equation for both the forward ordinary differential equation 510 and the reverse ordinary differential equation 512. Specifically, the base equation is:

$$\frac{d}{dt} x ( t ) = {f}_{θ} ( x ( t ) , t )$$

where x is a conformation for the query compound and f_{θ} is a neural network parameterized on e (e.g., the generative thermodynamics neural network of FIG. 1, 2, 3, or 4), and t is a certain time (e.g., a time step).

For training, the thermodynamics binding system 100 integrates over time the forward ODE which results in a sample from the target distribution Pt (e.g., which will become a learned Boltzmann distribution). This training process is supervised by the energy function of the resulting conformation utilizing an inverse Kullback-Leibler loss function.

When solving the forward ordinary differential equation 510, the thermodynamics binding system 100 relies on the principle that probability is conserved (e.g., the probabilities from the base distribution should add to 1) to determine each of the conformations. Accordingly, the thermodynamics binding system 100 manipulates the base equation into the following:

$$log ( {p}_{b} ( x ( t ) ) ) = log ( {p}_{t} ( x ( t ) ) ) - \frac{δ log ( {p}_{b} ( x ( t ) ) )}{δ t}$$

$$log ( {p}_{b} ( x ( t ) ) ) = log ( {p}_{t} ( x ( t ) ) ) - log \vert det \frac{δ f}{δ x ( t )} \vert$$

where x is the initial conformation p_{b} is the known, base distribution. Because probability is conserved, this means that the probability of the sample x in the base distribution p_{b} should be equal to the probability of sample x in the target pt minus the change of probability that it suffered during the transformation.

Further, the thermodynamics binding system 100 can apply an instantaneous change of variable to represent the forward ordinary differential equation 510 as:

$$log ( {p}_{b} ( x ( t ) ) ) = log ( {p}_{t} ( x ( t ) ) ) - tr ( \frac{δ f}{δ x ( t )} )$$

Further, the thermodynamics binding system 100 can estimate pt in the following way:

$${p}_{t} ( x ) = \frac{1}{Z} {e}^{- β E}$$

$$log ( {p}_{t} ( x ) ) = - β E - log ( Z )$$

$$log ( {p}_{t} ( x ) ) = - β E - C$$

Where E is the energy of the system, which can be calculated with a force field model (e.g., the force field model 332 of FIG. 3), β is the thermodynamic-beta, β=1/k_{B}T, and Z is the partition function which becomes a constant C. Accordingly, the final Kullback-Leibler loss becomes

$$K {L}_{( {p}_{t} \vert \vert {p}_{b} )} = - β E - tr ( \frac{δ f}{δ x ( t )} )$$

Indeed, the thermodynamics binding system 100 can map E to an energy distribution (e.g., a Boltzmann distribution) of the first compound 502.

The thermodynamics binding system 100 can solve the forward ordinary differential equation 510 (e., perform the base-to-energy distribution transformation process) for each of the predicted series of conformations 506 until the thermodynamics binding system 100 determines the binding conformation 508.

Upon determining the binding conformation 508, the thermodynamics binding system 100 can solve the reverse ordinary differential equation 512 to determine the binding metric. Indeed, the thermodynamics binding system 100 can estimate the probability for each sample of the target conformation, that is, for each bound or unbound conformation of the ligand. Specifically, the thermodynamics binding system 100 can integrate the reverse ordinary differential equation 512 over time in the following manner:

$$log ( {p}_{t} ( x ( t ) ) ) = log ( {p}_{b} ( x ( t ) ) ) + tr ( \frac{δ f}{δ x ( t )} )$$

$${p}_{t} bound = {\int }_{{x}_{bound}}^{{x}_{unbound}} log ( {p}_{b} ( x ( t ) ) ) + tr ( \frac{δ f}{δ x ( t )} )$$

By performing the reverse ordinary differential equation 512 (e.g., performing the energy-to-base distribution transformation process) for each of the predicted series of conformations 506, the thermodynamics binding system 100 can determine corresponding binding probabilities for the predicted series of conformations 506. Indeed, when determining the corresponding binding probabilities for the predicted series of conformations 506, in some implementations the thermodynamics binding system 100 once again relies on the assumption that probability is conserved (e.g., the probability of the energy distribution should add to 1). Specifically, the thermodynamics binding system 100 can utilize the sum of the corresponding binding probabilities to determine the binding metric by determining the probability of all of the bound states of the first compound 502.

Although not illustrated in FIG. 5, in some embodiments, the thermodynamics binding system 100 can receive a binding conformation 508 for the first compound 502 (e.g., as opposed to solving the forward ordinary differential equation 510 to determine the binding conformation 508), and can utilize the reverse ordinary differential equation 512 to determine the binding metric by determining the probability of all of the bound states for the first compound 502.

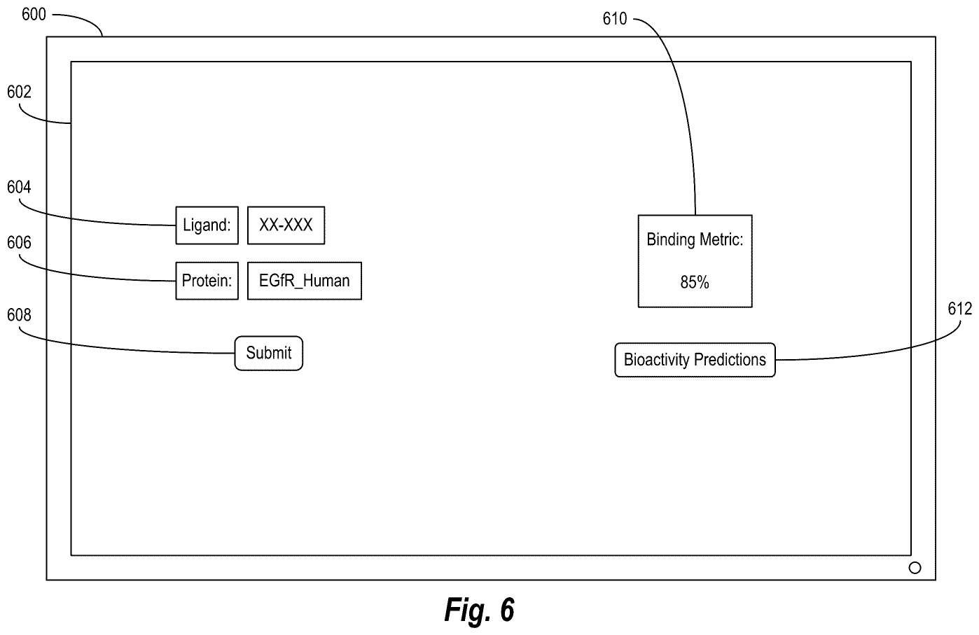

As previously mentioned, the thermodynamics binding system 100 can provide the binding metric in a user interface of a client device. FIG. 6 shows the thermodynamics binding system 100 providing the binding metric for a ligand and a protein in a user interface of a client device.

As shown in FIG. 6, the thermodynamics binding system 100 can receive a binding query for a ligand (e.g., a query compound) and a protein (e.g., a target protein) in a user interface 602 of a client device 600. Specifically, the thermodynamics binding system 100 can receive an input for the ligand through a first user-interface element 604. Additionally, the thermodynamics binding system 100 can receive an input for the protein through a second user-interface element 606. The ligand and protein can each respectively be selected by a user account from a drop-down menu, input by the user account, or a combination thereof. In some embodiments, the thermodynamics binding system 100 can provide suggestions for the ligand via the first user-interface element 604, the protein via the second user-interface element 606, and/or select the ligand and protein autonomously. Moreover, the thermodynamics binding system 100 can generate a third user-interface element 608 selectable to submit the binding query for the ligand and the protein.

Responsive to receiving an interaction with the third user-interface element 608 selectable to submit the binding query for the ligand and the protein, the thermodynamics binding system 100 can generate a fourth user-interface element 610 to display the binding metric in the user interface 602. Additionally, the thermodynamics binding system 100 can generate a fifth user-interface element 612 selectable to view the bioactivity predictions for the binding query according to the binding metric. In some embodiments, the fifth user-interface element 612 can be a drop-down menu. In other embodiments, the fifth user-interface element 612 can receive a specific input for a bioactivity prediction from a user account.

For example, in one or more embodiments, the thermodynamics binding system 100 can generate the bioactivity prediction selectable in the fifth user-interface element 612 by utilizing the binding metric and/or the binding conformation to identify potential compounds related to a target gene for treating a disease. The thermodynamics binding system 100 can then analyze compound features and determine a likelihood that the one or more potential compounds can be developed into treatments for the disease. To illustrate, the thermodynamics binding system 100 can initiate a compound program analysis based on the binding metric.

Indeed, the thermodynamics binding system 100 can utilize the binding metric to identify an anchor compound or anchor gene from the one or more promising potential compounds and/or genes. Upon determination of the one or more promising potential compounds and/or genes, the molecular graph prediction system can determine a program rating for the anchor compound and/or the anchor gene. For example, the thermodynamics binding system 100 can identify a protein that corresponds to a gene/disease of interest. The thermodynamics binding system 100 can generate binding metrics for the protein for a plurality of compounds. The thermodynamics binding system 100 can select a compound from the plurality of compounds to pursue as part of a compound program analysis.

In some embodiments, the thermodynamics binding system 100 can utilize the program rating to initiate a compound program analysis by initiating an industrial program generation (IPG) process. To illustrate, the molecular graph prediction system can utilize the IPG process to identify various components and/or requirements to develop the anchor compound into an advanced treatment for the disease. Specifically, the molecular graph prediction system can initiate the IPG process to identify information such as statistically strong connections in a biological map to patient-informed phenotypes, Trekseq confirmation (e.g., confirming anchor compound and anchor gene relationships utilizing transcriptomics), Structure-Activity Relationships (SAR) confidence, among others. Moreover, the molecular graph prediction system can utilize the program rating to initiate an industrialized compound generation (ICG) process to apply steps subsequent to the IPG process. For example, the molecular graph prediction system can utilize the ICG process to test the anchor compound with various analytical tests (e.g., SAR screens), or to identify other potential compounds related to the anchor compound for use in the treatment of the disease.

In one or more embodiments, the thermodynamics binding system 100 system can determine to utilize a program prediction as part of generating a program rating for initiation compound exploration programs, as described in U.S. patent application Ser. No. 18/521,910, titled “UTILIZING BIOLOGICAL MACHINE LEARNING REPRESENTATIONS AND A LANGUAGE MACHINE LEARNING MODEL FOR INITIATING COMPOUND EXPLORATION PROGRAMS,” which is incorporated by reference herein in its entirety.

Additionally, in one or more embodiments, the thermodynamics binding system 100 can generate the bioactivity prediction selectable in the fifth user-interface element 612 by utilizing a unique proteome fingerprint of the query compound to generate bioactivity results for the query compound. Indeed, the thermodynamics binding system 100 can determine, according to the binding metric, to extract the fingerprint from the query compound, the binding conformation, or the predicted series of conformations. The thermodynamics binding system 100 can utilize the fingerprint to generate ADMET predictions (e.g., molecular property predictions such as blood brain barrier properties) for query compounds. Moreover, the thermodynamics binding system 100 can utilize the binding metric and/or the fingerprint to generate biological perturbation predictions for the query compound and can display the biological perturbation predictions in the fifth user-interface element 612.

In one or more embodiments, the thermodynamics binding system 100 can determine to utilize the binding metric to generate a unique proteome fingerprint indicating query compound interactions within a compound-protein and utilize the fingerprint to generate predicted target bioactivity results for the query compound, as described in U.S. patent application Ser. No. 18/505,754, titled “UTILIZING COMPOUND-PROTEIN MACHINE LEARNING REPRESENTATIONS TO GENERATE BIOACTIVITY PREDICTIONS,” which is incorporated by reference herein in its entirety

The thermodynamics binding system 100 can thus improve user interfaces and reduce user interactions and computer resources relative to conventional systems. Indeed, by utilizing the graphical user interface described in FIG. 6, the thermodynamics binding system 100 can generate binding metrics, initiate a compound program analysis, and/or generate bioactivity predictions with a limited number of user interactions and user interfaces. Accordingly, the thermodynamics binding system 100 can significantly improve the efficiency of implementing computing devices and systems.

Additional detail regarding the molecular graph prediction system environment will now be provided with reference to FIG. 7. In particular, FIG. 7 illustrates a schematic diagram of a system environment in which the molecular graph prediction system can operate in accordance with one or more embodiments.

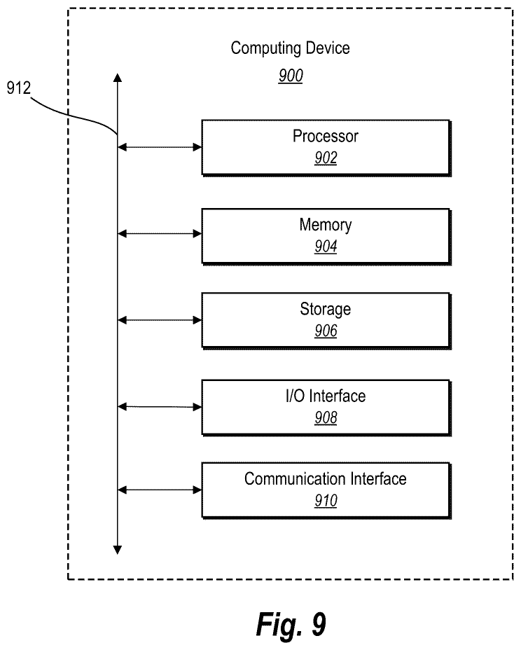

As shown in FIG. 7, the environment includes server(s) 700 (which includes a tech-bio exploration system 702 and the thermodynamics binding system 100), dedicated machine learning device(s) 714, a network 708, client device(s) 710 and administrator device(s) 712. As further illustrated in FIG. 7, the various computing devices within the environment can communicate via the network 708. Although FIG. 7 illustrates the thermodynamics binding system 100 being implemented by a particular component and/or device within the environment, the thermodynamics binding system 100 can be implemented, in whole or in part, by other computing devices and/or components in the environment (e.g., the additional device(s)). Additional description regarding the illustrated computing devices is provided with respect to FIG. 9 below.

As shown in FIG. 7, the server(s) 700 (e.g., one or more local servers operated by a particular entity) can include the tech-bio exploration system 702. In some embodiments, the tech-bio exploration system 702 can determine, store, generate, and/or display tech-bio information including maps of biology, experiments from various sources, and/or machine learning tech-bio predictions. For instance, the tech-bio exploration system 702 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 702 provides an environment for operating, executing, and managing complex drug discovery pipelines.

For instance, the tech-bio exploration system 702 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 702 can generate or determine a variety of predictions and inter-relationships for improving treatments/interventions.

To illustrate, the tech-bio exploration system 702 can generate maps of biology indicating biological inter-relationships or similarities between these various input signals to discover potential new treatments as part of the complex compound discovery process. For example, the tech-bio exploration system 702 can utilize machine learning and/or maps of biology to identify a similarity between a first gene associated with disease treatment and a second gene previously unassociated with the disease based on a similarity in resulting phenotypes from gene knockout experiments. The tech-bio exploration system 702 can then identify new treatments based on the gene similarity (e.g., by targeting compounds the impact the second gene). Similarly, the tech-bio exploration system 702 can analyze signals from a variety of sources (e.g., protein interactions, or invivo experiments) to predict efficacious treatments based on various levels of biological data.

The tech-bio exploration system 702 can generate GUIs comprising dynamic user interface elements to convey tech-bio information and receive user input for intelligently exploring tech-bio information. Indeed, as mentioned above, the tech-bio exploration system 702 can generate GUIs displaying different maps of biology that intuitively and efficiently express complex interactions between different biological systems for identifying improved treatment solutions. Furthermore, the tech-bio exploration system 702 can also electronically communicate tech-bio information between various computing devices.

As shown in FIG. 7, the tech-bio exploration system 702 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 702 collects, manages, and transmits data across a variety of different entities, accounts, and devices. In some cases, the tech-bio exploration system 702 is a network system that facilitates access to (and analysis of) tech-bio information within a centralized operating system. Indeed, the tech-bio exploration system 702 can link data from different network-based research institutions to generate and analyze maps of biology.

As shown in FIG. 7, the tech-bio exploration system 702 can include a system that comprises the thermodynamics binding system 100 that generates, stores, manages, transmits data pertaining to the generation of binding conformations of a query compound and the utilization of that binding conformation to generate a binding metric for the query compound. The binding metric can subsequently be used to generate biological activity predictions for the query compound. For example, in context of the above description for the tech-bio exploration system 702, in some embodiments the tech-bio exploration system 702 further utilizes the thermodynamics binding system 100 to enhance the coordination between various groups involved in the drug discovery process. For instance, the thermodynamics binding system 100 works in tandem with the tech-bio exploration system 702 to generate binding conformations, generate binding metrics from the binding conformations, utilize the binding metrics to generate biological activity predictions, transmit the biological activity predictions to one or more devices, and initiate one or more downstream model predictions or processes.

As also illustrated in FIG. 7, the environment includes the client device(s) 710. As mentioned above, the client device(s) 710 can be involved in the process of drug discovery. Thus, for example, the client device(s) 710 can coordinate/manage a first stage of generating binding conformation of a query compound. Moreover, the client device(s) 710 can coordinate/manage a second stage such as determining a binding metric for binding conformation of the query compound and a target protein. Further, the client device(s) 710 can coordinate/manage a third stage of utilizing the binding metric to generate a biological prediction to generate one or more additional predictions or initiate one or more programs (IPG or ICG).

To illustrate, the client device(s) 710 can include computing devices that implement or manage a compound program generation stage of a compound discovery process. Similarly, the client device(s) 710 can include computing devices that implement or manage a compound lead generation stage and the client device(s) 710 can include computing devices that implement or manage a compound/dose selection stage. For example, the thermodynamics binding system 100 can receive one or more requests to utilize the dedicated machine learning device(s) 714 to determine a binding metric from a binding conformation of a query compound. For instance, the thermodynamics binding system 100 can receive additional requests from the client device(s) 710 that include generating the biological activity predictions from the binding metric.

In some embodiments, the environment also includes additional device(s). For example, the thermodynamics binding system 100 can utilize the additional device(s) to further operate and manage the completion of complex drug discovery pipelines. For instance, the additional device(s) include experimental device(s) and analytical device(s). Further, in some instances, the additional device(s) also include the computing devices discussed below in FIG. 9.

Furthermore, in one or more implementations, the client device(s) 710 include a client application. The client application can include instructions that (upon execution) cause the client device(s) 710 to perform various actions. For example, a user of a user account can interact with the client application on the client device(s) 710 to execute experiments or other multi-faceted processes and to further access tech-bio information, initiate a request for a binding conformation, a binding metric, or a biological activity prediction. For instance, in some embodiments the thermodynamics binding system 100 receives a request to generate a binding conformation for a query compound and a target protein, and in response generates the binding conformation and returns the binding conformation to the client device(s) 710. In some instances, the transmittal of the binding conformation to the client device(s) 710 causes the client device(s) 710 to execute an action (e.g., generate a binding metric or generate a downstream model prediction).

As shown, the environment can also include dedicated machine learning device(s) 714. For example, the dedicated machine learning device(s) 714 can include computing devices or virtual machines dedicated to training or implementing large-scale machine learning models. For example, the dedicated machine learning device(s) 714 can generate machine learning predictions and/or embeddings based on digital biological data (e.g., digital images of phenotypes resulting from different perturbations or compound-protein interactions from compound features). As shown, the dedicated machine learning device(s) 714 includes a generative thermodynamics neural network 716. Thus, the thermodynamics binding system 100 interacts with the dedicated machine learning device(s) 714 to generate binding metrics from binding conformations of query compounds and generate biological activity predictions for the query compounds utilizing the binding metrics.

The environment can also include experimental device(s). For example, the tech-bio exploration system 702 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 702 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the thermodynamics binding system 100 generates binding conformation, determines the binding metric, and further determines to employ or utilize one or more experimental devices (e.g., to initiate one or more experiments based on the binding conformations or the binding metrics).

As further shown in FIG. 7, the environment includes the network 708. As mentioned above, the network 708 can enable communication between components of the environment. In one or more embodiments, the network 708 may include a suitable network and may communicate using a various number of communication platforms and technologies suitable for transmitting data and/or communication signals, examples of which are described with reference to FIG. 9. Furthermore, although FIG. 7 illustrates computing devices communicating via the network 708, the various components of the environment can communicate and/or interact via other methods (e.g., communicate directly).

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

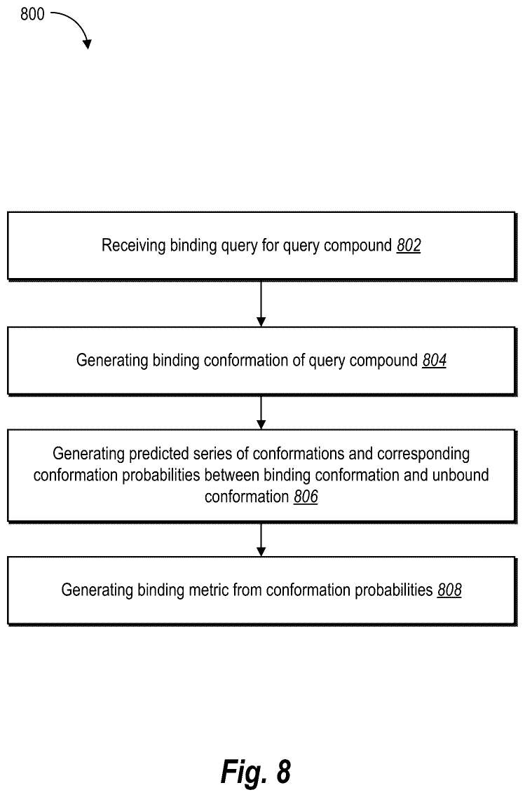

FIGS. 1-7, the corresponding text, and the examples provide a number of different systems, methods, and non-transitory computer readable media for generating a binding conformation for a query compound and a target protein and utilizing the binding conformation to determine a binding metric representative of the binding metric representative of the binding interaction between the target protein and the query compound. In addition to the foregoing, embodiments can also be described in terms of flowcharts comprising acts for accomplishing a particular result. For example, FIG. 8 illustrates a flowchart of an example sequence of acts in accordance with one or more embodiments.

While FIG. 8 illustrates acts according to some embodiments, alternative embodiments may omit, add to, reorder, and/or modify any of the acts shown in FIG. 8. The acts of FIG. 8 can be performed as part of a method (e.g., a computer-implemented method). Alternatively, a non-transitory computer readable medium can comprise instructions, that when executed by one or more processors (e.g., at least one processor), cause a computing device to perform the acts of FIG. 8. In still further embodiments, a system can perform the acts of FIG. 8. Additionally, the acts described herein may be repeated or performed in parallel with one another or in parallel with different instances of the same or other similar acts.

FIG. 8 illustrates an example series of acts 800 for determining a binding metric for a query compound and target protein. The series of acts 800 can include acts 802-808 of receiving a binding query for a query compound; generating a binding conformation of the query compound; generating a predicted series of conformations and corresponding conformation probabilities between binding conformations and an unbound conformation; and generating a binding metric from the conformation probabilities.

For example, in one or more embodiments, acts 802-808 include receiving, from a computing device, a binding query for a query compound and a target protein; generating, utilizing a generative thermodynamics neural network in a base-to-energy distribution transformation process, a binding conformation of the query compound corresponding to a binding interaction between the query compound and the target protein; generating, utilizing the generative thermodynamics neural network in an energy-to-base distribution transformation process, a predicted series of conformations and corresponding conformation probabilities between the binding conformation and an unbound conformation of the query compound; and generating, in response to the binding query from the computing device, a binding metric representative of the binding interaction between the target protein and the query compound from the conformation probabilities of the predicted series of conformations.

In one or more implementations, the series of acts 800 include training the generative thermodynamics neural network by: sampling, from a base distribution, an initial conformation of a training compound; and generating, from the initial conformation of the training compound, a binding conformation of the training compound relative to a training protein.

In addition, in one or more implementations, the series of acts 800 include training the generative thermodynamics neural network by determining a measure of energy corresponding to the binding conformation; and modifying parameters of the generative thermodynamics neural network based on the measure of energy.

Further, in some implementations, the series of acts 800 include determining the measure of energy by utilizing a force field model to generate a force field value based on the binding conformation of the training compound in binding with the training protein.

In one or more implementations, the series of acts 800 further includes generating the binding conformation of the query compound by: sampling an initial conformation of the query compound from the base distribution; and generating, at a first time step utilizing the generative thermodynamics neural network, a first conformation of the query compound from the initial conformation and the target protein, wherein the first conformation includes at least one of a first translation of the query compound, a first rotation of the query compound, or a first set of modified dihedral angles of the query compound.

In addition, in some implementations, the series of acts 800 includes generating the conformation of the query compound by generating, at an additional time step utilizing the generative thermodynamics neural network, the binding conformation of the query compound based on the modified conformation and the target protein.

Additionally, in one or more embodiments, the base-to-energy distribution transformation process includes an ordinary differential equation that utilizes the generative thermodynamics neural network over a series of time steps to transform the base distribution for the query compound to the energy distribution for the query compound.

Further, in some implementations, the energy-to-base distribution transformation process includes a reverse ordinary differential equation integrated over time steps of the generative thermodynamics neural network to determine the binding metric.

In some implementations, the generative thermodynamics neural network is trained to map a base distribution of query compound conformation to an energy distribution for query compound conformations relative to target proteins.

FIG. 9 illustrates a block diagram of an example computing device 900 that may be configured to perform one or more of the processes described above. One will appreciate that one or more computing devices, such as the computing device 900 may represent the computing devices described above. In one or more embodiments, the computing device 900 may be a mobile device (e.g., a mobile telephone, a smartphone, a PDA, a tablet, a laptop, a camera, a tracker, a watch, a wearable device, etc.). In some embodiments, the computing device 900 may be a non-mobile device (e.g., a desktop computer or another type of client device). Further, the computing device 900 may be a server device that includes cloud-based processing and storage capabilities.

As shown in FIG. 9, the computing device 900 can include one or more processor(s) 902, memory 904, a storage device 906, input/output interfaces 908 (or “I/O interfaces 908”), and a communication interface 910, which may be communicatively coupled by way of a communication infrastructure (e.g., bus 912). While the computing device 900 is shown in FIG. 9, the components illustrated in FIG. 9 are not intended to be limiting. Additional or alternative components may be used in other embodiments. Furthermore, in certain embodiments, the computing device 900 includes fewer components than those shown in FIG. 9. Components of the computing device 900 shown in FIG. 9 will now be described in additional detail.

In particular embodiments, the processor(s) 902 includes hardware for executing instructions, such as those making up a computer program. As an example, and not by way of limitation, to execute instructions, the processor(s) 902 may retrieve (or fetch) the instructions from an internal register, an internal cache, memory 904, or a storage device 906 and decode and execute them.

The computing device 900 includes memory 904, which is coupled to the processor(s) 902. The memory 904 may be used for storing data, metadata, and programs for execution by the processor(s). The memory 904 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 904 may be internal or distributed memory.

The computing device 900 includes a storage device 906 includes storage for storing data or instructions. As an example, and not by way of limitation, the storage device 906 can include a non-transitory storage medium described above. The storage device 906 may include a hard disk drive (HDD), flash memory, a Universal Serial Bus (USB) drive or a combination these or other storage devices.

As shown, the computing device 900 includes one or more I/O interfaces 908, which are provided to allow a user to provide input to (such as user strokes), receive output from, and otherwise transfer data to and from the computing device 900. These I/O interfaces 908 may include a mouse, keypad or a keyboard, a touch screen, camera, optical scanner, network interface, modem, other known I/O devices or a combination of such I/O interfaces 908. The touch screen may be activated with a stylus or a finger.

The I/O interfaces 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 908 are configured to provide graphical data to a display for presentation to a user. The graphical data may be representative of one or more graphical user interfaces and/or any other graphical content as may serve a particular implementation.

The computing device 900 can further include a communication interface 910. The communication interface 910 can include hardware, software, or both. The communication interface 910 provides one or more interfaces for communication (such as, for example, packet-based communication) between the computing device and one or more other computing devices or one or more networks. As an example, and not by way of limitation, communication interface 910 may include a network interface controller (NIC) or network adapter for communicating with an Ethernet or other wire-based network or a wireless NIC (WNIC) or wireless adapter for communicating with a wireless network, such as a WI-FI. The computing device 900 can further include a bus 912. The bus 912 can include hardware, software, or both that connects components of computing device 900 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 900 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 702 may include a variety of servers, sub-systems, programs, modules, logs, and data stores. In particular embodiments, the tech-bio exploration system 702 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 702 may also include suitable components such as network interfaces, security mechanisms, load balancers, failover servers, management-and-network-operations consoles, other suitable components, or any suitable combination thereof. In particular embodiments, the tech-bio exploration system 702 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 702 and one or more client devices. An action logger may be used to receive communications from a web server about a user's actions on or off the tech-bio exploration system 702. In conjunction with the action log, a third party-content-object log may be maintained of user exposures to third party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 702. A privacy setting of a user determines how particular information associated with a user can be shared. The authorization server may allow users to opt in to or opt out of having their actions logged by the tech-bio exploration system 702 or shared with other systems, such as, for example, by setting appropriate privacy settings. Third party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
receiving, from a computing device, a binding query for a query compound and a target protein;
generating, utilizing a generative thermodynamics neural network in a base-to-energy distribution transformation process, a binding conformation of the query compound corresponding to a binding interaction between the query compound and the target protein;
generating, utilizing the generative thermodynamics neural network in an energy-to-base distribution transformation process, a predicted series of conformations and corresponding conformation probabilities between the binding conformation and an unbound conformation of the query compound; and
generating, in response to the binding query from the computing device, a binding metric representative of the binding interaction between the target protein and the query compound from the conformation probabilities of the predicted series of conformations.

2. The computer-implemented method of claim 1, further comprising:
training the generative thermodynamics neural network by:
sampling, from a base distribution, an initial conformation of a training compound; and
generating, from the initial conformation of the training compound, a binding conformation of the training compound relative to a training protein.

3. The computer-implemented method of claim 2, further comprising:
determining a measure of energy corresponding to the binding conformation; and
modifying parameters of the generative thermodynamics neural network based on the measure of energy.

4. The computer-implemented method of claim 3, further comprising determining the measure of energy by utilizing a force field model to generate a force field value based on the binding conformation of the training compound in binding with the training protein.

5. The computer-implemented method of claim 1, further comprising generating the binding conformation of the query compound by:
sampling an initial conformation of the query compound from a base distribution; and
generating, at a first time step utilizing the generative thermodynamics neural network, a first conformation of the query compound from the initial conformation and the target protein, wherein the first conformation comprises at least one of a first rotation of the query compound, a first translation of the query compound, or a first set of modified dihedral angles of the query compound.

6. The computer-implemented method of claim 5, further comprising generating the binding conformation of the query compound by generating, at an additional time step utilizing the generative thermodynamics neural network, the binding conformation of the query compound based on the first conformation and the target protein.

7. The computer-implemented method of claim 6, wherein the base-to-energy distribution transformation process comprises an ordinary differential equation that utilizes the generative thermodynamics neural network over a series of time steps to transform the base distribution for the query compound to an energy distribution for the query compound.

8. The computer-implemented method of claim 7, wherein the energy-to-base distribution transformation process comprises a reverse ordinary differential equation integrated over time steps of the generative thermodynamics neural network to determine the binding metric.

9. The computer-implemented method of claim 1, wherein the generative thermodynamics neural network is trained to map a base distribution of query compound conformations to an energy distribution for query compound conformations relative to target proteins.

10. A system comprising:
at least one processor; and
at least one non-transitory computer-readable storage medium storing instructions that, when executed by the at least one processor, cause the system to:
receive, from a computing device, a binding query for a query compound and a target protein;
generate, utilizing a generative thermodynamics neural network in a base-to-energy distribution transformation process, a binding conformation of the query compound corresponding to a binding interaction between the query compound and the target protein;
generate, utilizing the generative thermodynamics neural network in an energy-to-base distribution transformation process, a predicted series of conformations and corresponding conformation probabilities between the binding conformation and an unbound conformation of the query compound; and
generate, in response to the binding query from the computing device, a binding metric representative of the binding interaction between the target protein and the query compound from the conformation probabilities of the predicted series of conformations.

11. The system of claim 10, further comprising instructions that, when executed by the at least one processor, cause the system to:
train the generative thermodynamics neural network by:
sampling, from a base distribution, an initial conformation of a training compound; and
generating, from the initial conformation of the training compound, a binding conformation of the training compound relative to a training protein.

12. The system of claim 11, further comprising instructions, that, when executed by the at least one processor, cause the system to:
determine a measure of energy corresponding to the binding conformation; and
modify parameters of the generative thermodynamics neural network based on the measure of energy.

13. The system of claim 12, further comprising instructions that, when executed by the at least one processor, cause the system to determine the measure of energy by utilizing a force field model to generate a force field value based on the binding conformation of the training compound in binding with the training protein.

14. The system of claim 10, further comprising instructions that, when executed by the at least one processor, cause the system to generate the binding conformation of the query compound by:
sampling an initial conformation of the query compound from a base distribution; and
generating, at a first time step utilizing the generative thermodynamics neural network, a first conformation of the query compound from the initial conformation and the target protein, wherein the first conformation comprises at least one of a first rotation of the query compound, a first translation of the query compound, or a first set of modified dihedral angles of the query compound.

15. The system of claim 14, further comprising instructions that, when executed by the at least one processor, cause the system to generate the binding conformation of the query compound by generating, at an additional time step utilizing the generative thermodynamics neural network, a binding conformation of the query compound based on the first conformation and the target protein.

16. A non-transitory computer-readable medium storing instructions that, when executed by at least one processor, cause a computing device to:
receive, from a computing device, a binding query for a query compound and a target protein;
generate, utilizing a generative thermodynamics neural network in a base-to-energy distribution transformation process, a binding conformation of the query compound corresponding to a binding interaction between the query compound and the target protein;
generate, utilizing the generative thermodynamics neural network in an energy-to-base distribution transformation process, a predicted series of conformations and corresponding conformation probabilities between the binding conformation and an unbound conformation of the query compound; and
generate, in response to the binding query from the computing device, a binding metric representative of the binding interaction between the target protein and the query compound from the conformation probabilities of the predicted series of conformations.

17. The non-transitory computer-readable medium of claim 16, further comprising instructions that, when executed by the at least one processor, cause the computing device to:
train the generative thermodynamics neural network by:
sampling, from a base distribution, an initial conformation of a training compound; and
generating, from the initial conformation of the training compound, a binding conformation of the training compound relative to a training protein.

18. The non-transitory computer-readable medium of claim 17, further comprising instructions that, when executed by the at least one processor, cause the computing device to:
determine a measure of energy corresponding to the binding conformation; and
modify parameters of the generative thermodynamics neural network based on the measure of energy.

19. The non-transitory computer-readable medium of claim 18, further comprising instructions that, when executed by the at least one processor, cause the computing device to determine the measure of energy by utilizing a force field model to generate a force field value based on the binding conformation of the training compound in binding with the training protein.

20. The non-transitory computer-readable medium of claim 16, further comprising instructions that, when executed by the at least one processor, cause the computing device to generate the binding conformation of the query compound by:
sampling an initial conformation of the query compound from a base distribution; and
generating, at a first time step utilizing the generative thermodynamics neural network, a first conformation of the query compound from the initial conformation and the target protein, wherein the first conformation comprises at least one of a first rotation of the query compound, a first translation of the query compound, or a first set of modified dihedral angles of the query compound.
